(19) United States
(12) Patent Application Publication
(10) Pub. No.: US 2002/0055773 A1
(43) Pub. Date: May 9, 2002

(54) POLYMER HEART VALVE WITH INSERT MOLDED FABRIC SEWING CUFF

(76) Inventors: LOUIS A. CAMPBELL, AUSTIN, TX (US); RIYAD E. MOE, AUSTIN, TX (US); EDWARD SARNOWSKI, AUSTIN, TX (US); JAMES C. CAFFEY, MARBLE FALLS, TX (US)

Correspondence Address: TIMOTHY L SCOTT, SULZER MEDICA USA INC, 3 EAST GREENWAY PLAZA, SUITE 1600, HOUSTON, TX, 77046

(21) Appl. No.: 09/352,415

(22) Filed: Jul. 12, 1999

(51) Int. Cl. A61F 2/24

(52) U.S. Cl. 623/2.4

(57) ABSTRACT

A flexible leaflet heart valve includes an annular body and an annular base portion connected to an end of the body. A fabric member is molded into the base portion to form an uninterrupted annular fabric band around the periphery of the base portion. The fabric band may include a first portion molded into the base and a second portion extending from the base. Alternatively, the fabric band may be entirely encapsulated in the base. The valve fabric can be sutured directly to patient tissue, or the valve fabric can be sutured to an adjacent sewing cuff which is sutured to patient tissue.

### BACKGROUND

The disclosures herein relate generally to prosthetic heart valves and more particularly to a heart valve having an integrally molded fabric sewing cuff.

Mechanical valves use interference lockwires or cinched down suture rings to attach sewing cuffs thereto. An example of such valves is found in U.S. Pat. No. 5,071,431 which discloses a suture ring for a mechanical heart valve comprised of essentially three parts: a stiffening ring which fits over an outer surface of the heart valve; and knit fabric sewing cuff attached to the stiffening ring, and a locking ring for securing the stiffening ring to the heart valve. The locking ring is generally crescent-shaped in cross-section and has a split ring configuration. Radial expansion or contraction is a first mode of deformation for the locking ring. The stiffening ring and the valve body can be locked together because the locking ring is capable of deforming in a second mode with respect to thickness of the ring. The locking ring deforms by a slight flattening of the cresent cross-section of the ring.

U.S. Pat. No. 5,397,346 discloses a prosthetic mechanical heart valve with a suture ring and stiffening ring combination which minimizes the radial thickness of the sewing ring. The valve comprises an annular stiffening ring captured between upper and lower rings which are sewn into a knit fabric tube. The entire assembly of sewing ring and stiffening ring is then assembled as a unit onto the mechanical heart valve.

An elastomeric polymer valve, similar to a tissue valve, does not have a rigid enough structure for the interference lockwire technique use in mechanical valves, as mentioned above. For example, U.S. Pat. No. 5,562,729 discloses a multi-leaflet (usually trileaflet) heart valve composed of biocompatible polymer which simultaneously imitates the structure and dynamics of biological heart valves and avoids promotion of calcification. The valve includes a plurality of flexible leaflets dip cast on a mandrel, which leaflets are then bonded with a bonding agent to the interior surfaces of a plurality of struts on a metal-reinforced prosthetic stent. The leaflets open and close in response to the pumping action of the heart and, due to the design of the leaflets, fatigue resistance of the heart valve is high. The leaflets and the polymer components of the prosthetic are manufactured of biocompatible polymers exhibiting intrinsic calcification-resistant properties.

Tissue valves use sutures to attach the sewing cuff to the valve body. The suture technique is not preferred for polymer valves because passing a needle through the polymer may produce crack initiation points. For example, U.S. Pat. No. 4,364,127 discloses a prosthetic heart valve constructed of hemo-compatible materials that is anatomically and functionally similar to the natural aortic valve. The heart valve is a trileaflet type which has its formed leaflets heat set in a partially open position to reduce pressure required to open the leaflets in response to blood flowing therethrough.

U.S. Pat. No. 4,106,129 discloses a supported bioprosthetic heart valve in which the supporting stent is capable of annular deformation and also of limited perimetric expansion and contraction during heart operation. The stent includes a wire frame composed of a single flexible wire preformed to define inverted U-shaped commissure supports merging smoothly with arcuate portions connecting such supports.

U.S. Pat. No. 4,501,030 discloses a prosthetic heart valve including a frame having a plurality of commissure supports, a plurality of resilient supports, and a plurality of valve leaflets. The valve leaflets are attached to the resilient supports, and the resilient supports, respectively. When in use, the valve is subjected to forces which are used to clamp the valve leaflets between the resilient supports and the commissure supports to augment whatever other leaflet attachment techniques may be used.

One proposed solution includes a valve in which a sewing cuff with a rigid ring inside, is insert molded into a compression molded silicone valve. [refer to Roe article].

Therefore, what is needed is a fabric member insert molded into the periphery of a valve body so as to form an uninterrupted band of fabric around the valve.

### SUMMARY

One embodiment, accordingly, provides fabric which is insert molded into the valve body. To this end, a prosthetic heart valve includes a molded body having a base. The base has a peripheral portion and a fabric is molded into the body adjacent the peripheral portion.

A principal advantage of this embodiment is that the imbedded fabric functions as a tear stopping member which limits the growth of cracks from needle penetrations formed in the valve body during suturing of the valve to patient tissue or to a sewing cuff.

### BRIEF DESCRIPTION OF THE DRAWING FIGURES

FIG. 1 is an isometric view illustrating an embodiment of a prosthetic heart valve.

FIG. 2 is a top view illustrating an embodiment of the prosthetic heart valve of FIG. 1.

FIG. 3 is a partial view illustrating an embodiment of a peripheral portion of a prosthetic heart valve including a fabric molded therein.

FIG. 4 is a partial view illustrating an embodiment of a peripheral portion of a prosthetic heart valve including a fabric molded therein.

FIG. 5 is a partial view illustrating an embodiment of a peripheral portion of a prosthetic heart valve including a fabric molded therein and attached to a sewing cuff.

FIG. 6 is a partial view illustrating an embodiment of a peripheral portion of a prosthetic heart valve including a fabric molded therein and attached to a sewing cuff.

FIG. 7 is a partial view illustrating another embodiment of a sewing cuff molded into a prosthetic heart valve.

FIG. 8 is a partial view illustrating a further embodiment of a sewing cuff molded into a prosthetic heart valve.

### DETAILED DESCRIPTION

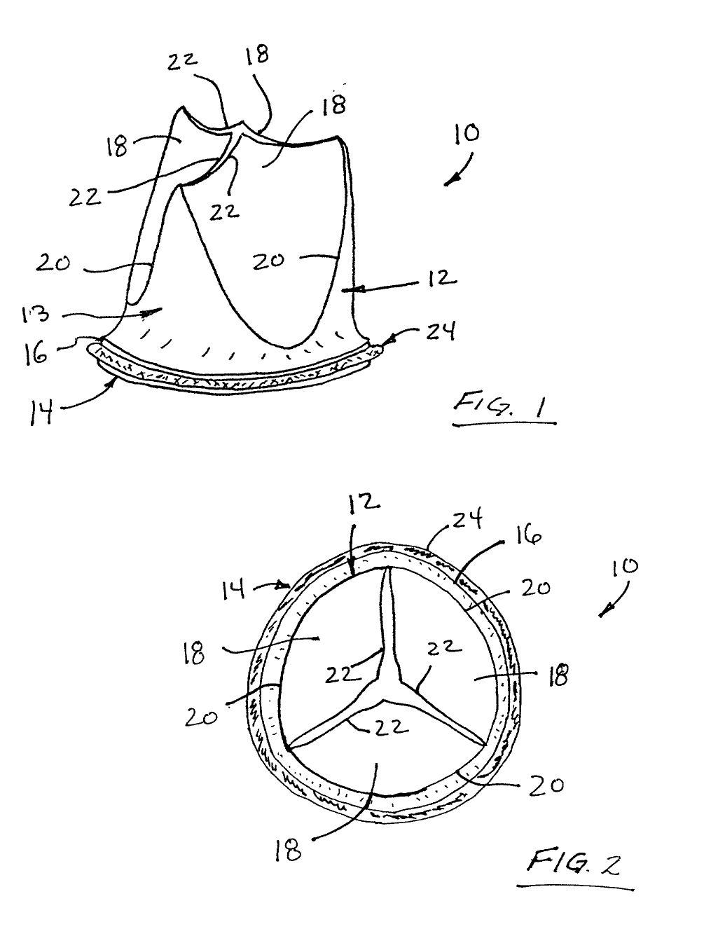

A prosthetic heart valve is generally designated 10 in FIGS. 1 and 2. Heart valve 10 is formed as a one-piece molded biocompatible polymer body 12 such as silicone or polyurethane and includes a generally annular peripheral wall 13 and an annular base 14. Base 14 includes an annular peripheral portion 16. Typically base 14 is sutured to an annulus of a natural heart valve (not shown). A plurality of flexible leaflets 18 are formed with the body 12 and extend from an attachment curve 20 to terminate at a free margin 22. A fabric member 24 is molded into the base 14 adjacent the annular peripheral portion 16 to form an uninterrupted annular band.

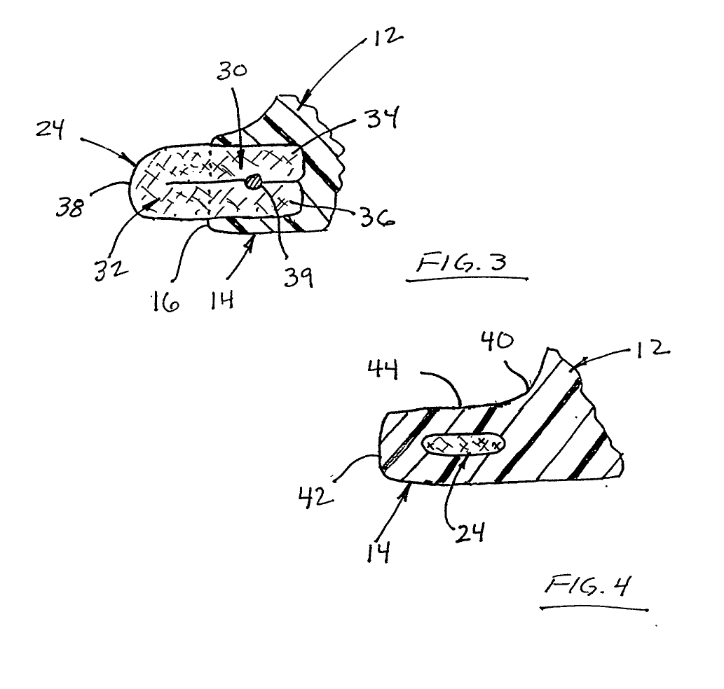

The fabric member 24 may be molded into base 14 in various configurations generally described as partially molded into base 14, FIG. 3, and completely imbedded or encapsulated in base 14, FIG. 4. Referring to FIG. 3, with base 14 extending radially from body 12, fabric 24 may include a first portion 30 molded into base 14. A second portion 32 may extend from annular peripheral portion 16 of base 14 to form an uninterrupted annular fabric band. The fabric member 24 may comprise a first end 34, a second end 36 and an intermediate portion 38 between the first end 34 and the second end 36. The first end and second ends are folded or doubled over and molded into base 14, and the intermediate portion 38 extends radially from annular peripheral portion 16 of base 14 to form an uninterrupted annular band around annular peripheral portion 16. Also, if desired, an annular reinforcing ring 39 may be molded into base 14 between the doubled-over first and second ends 34, 36, respectively.

Referring to FIG. 4, base 14 may include a first end 40 attached to body 12 and extending nearly to a terminal end 42. A mid-portion 44 of base 14 is between first end 40 and second end 42. Fabric 24 is encapsulated in mid-portion 44 of base 14 between first end 40 and terminal end 42 for forming an uninterrupted annular band within base 14.

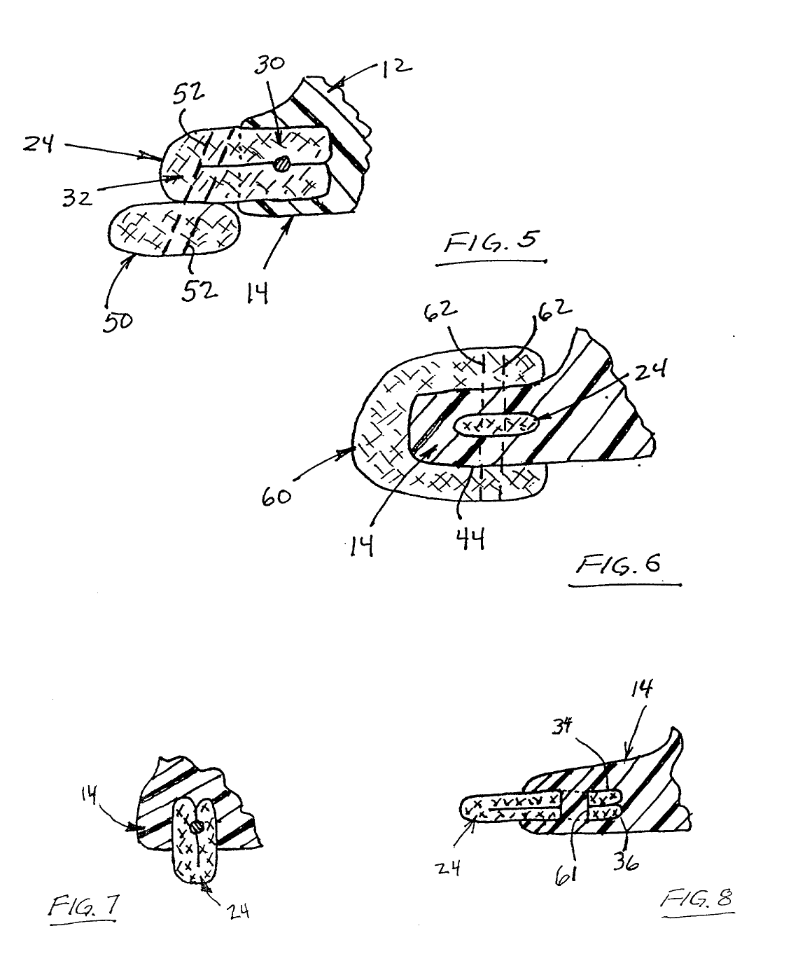

In FIG. 5, with base 14 extending from body 12, fabric 24 includes first portion 30 molded into base 14 and second portion 32 extending from base 14 to form an uninterrupted annular band. An annular sewing cuff 50 may be attached by stitches 52 or the like, to annular second portion 32 of fabric 24. Similarly, in FIG. 6, with fabric 24 encapsulated into mid-portion 44 of base 14, an annular sewing cuff 60 may be attached by stitches 62 or the like, to fabric 24.

In FIG. 7, the fabric member 24 may extend axially from the base 14 rather than radially as illustrated in FIG. 3. In FIG. 8, fabric member 24 extends radially from base 14. First and second ends 34 and 36 respectively, of fabric member 24 are doubled over and molded into base 14. Ends 34 and 36 include perforations 61 (only one of which is visible in FIG. 8) so that the polymer material can flow through each perforation 61 during the molding process to lock the fabric 24 into base 14.

In operation, a fabric member forms an uninterrupted annular band around the base portion of a molded prosthetic heart valve. The fabric member may be partially molded into the base so that a portion of the fabric member extends from the annular periphery of the base. Alternatively, the fabric member may be completely imbedded in the base portion and encapsulated therein. The fabric member may be directly attached to the annulus of a natural heart valve, or may be attached to a sewing cuff which is attached directly to the natural heart valve annulus.

As a result, one embodiment provides a prosthetic heart valve including a molded body having a base with a peripheral portion. A fabric is molded into the base adjacent the peripheral portion.

Another embodiment provides a molded prosthetic heart valve including a body having a peripheral portion and a plurality of leaflets attached thereto. A base portion is attached to an end of the body. A fabric is molded into the base portion.

A further embodiment provides a flexible leaflet heart valve including an annular body having an annular base portion connected to an end of the body. A fabric is molded into the base portion.

A still further embodiment provides a method of forming a flexible leaflet heart valve including forming an annular valve body including a plurality of flexible leaflets formed with a first end thereof. An annular base portion is connected to a second end of the body. A fabric member is embedded into the base portion to form an annular fabric band therewith.

As it can be seen, the principal advantages of these embodiments are that the embedded fabric functions as a tear stopping member which limits the growth of cracks from needle penetrations formed in the valve body during suturing of the valve. The fabric can be entirely encapsulated in a molded valve or a portion of the fabric can be molded into the valve leaving another portion of the fabric to extend from the valve. As a result, the valve fabric can be sutured directly to patient tissue, or alternatively, the valve fabric can be sutured to an adjacent sewing cuff which is sutured to patient tissue.

Although illustrative embodiments have been shown and described, a wide range of modification change and substitution is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

What is claimed is:

1. A prosthetic heart valve comprising:
a molded body including a base having a peripheral portion;
a fabric molded into the base adjacent the peripheral portion.

2. The valve as defined in claim 1 wherein the fabric is an uninterrupted annular band.

3. The valve as defined in claim 1 wherein a first portion of the fabric is embedded into the base and a second portion of the fabric forms an uninterrupted annular band extending from the peripheral portion.

4. The valve as defined in claim 1 wherein the fabric is entirely encapsulated only in the base.

5. The valve as defined in claim 1 further comprising a peripheral sewing cuff attached to the fabric.

6. The valve as defined in claim 5 wherein the fabric is an uninterrupted annular band.

7. The valve as defined in claim 5 wherein a first portion of the fabric is imbedded into the base and a second portion of the fabric forms an uninterrupted annular band extending from the base.

8. The valve as defined in claim 5 wherein the fabric is entirely embedded in the base.

9. A molded prosthetic heart valve comprising:
a body including a peripheral portion and a plurality of leaflets attached thereto;
a base portion attached to an end of the body; and
fabric molded into the base portion.

10. The valve as defined in claim 9 wherein the fabric member includes a first end, a second end and an intermediate portion.

11. The valve as defined in claim 10 wherein the first and second ends are molded into the base portion and the intermediate portion extends from the base portion.

12. The valve as defined in claim 11 wherein the intermediate portion forms an uninterrupted annular band around the base portion.

13. The valve as defined in claim 9 wherein the base portion forms an annular flange around the end of the body, the flange having a first end connected to the body and having a second terminal end.

14. The valve as defined in claim 13 wherein the fabric member is molded into the base between the first end and the second end.

15. A flexible leaflet heart valve comprising:
an annular body;
an annular base portion connected to an end of the body; and
a fabric member molded into the base portion.

16. The valve as defined in claim 15 wherein the fabric member includes a first end, a second end and an intermediate portion.

17. The valve as defined in claim 16 wherein the first and second ends are molded into the base portion and the intermediate portion extends from the base portion.

18. The valve as defined in claim 16 wherein the intermediate portion forms an uninterrupted band around the base portion.

19. The valve as defined in claim 15 wherein the base portion forms an annular flange around the end of the body, the flange having a first end connected to the body and having a second terminal end.

20. The valve as defined in claim 19 wherein the fabric member is molded into the base portion between the first end and the second end.

21. The valve as defined in claim 15 wherein the fabric member is an uninterrupted annular band.

22. The valve as defined in claim 15 wherein a first portion of the fabric member is embedded into the body and a second portion of the fabric member forms an uninterrupted annular band extending from the base portion.

23. The valve as defined in claim 15 wherein the fabric member is entirely encapsulated in the base portion.

24. The valve as defined in claim 15 further comprising a peripheral sewing cuff adjacent the base portion.

25. The valve as defined in claim 23 wherein the fabric member is an uninterrupted annular band.

26. The valve as defined in claim 15 wherein a portion of the fabric member is embedded into the base portion and a second portion of the fabric member extends from the base portion, the first portion of the fabric member including perforations formed therein for interlocking engagement with the base portion.

27. A method of forming a flexible leaflet heart valve comprising the steps of:
forming an annular valve body including a plurality of flexible leaflets formed with a first end thereof;
simultaneously forming an annular base portion with a second end of the body; and
positioning a fabric member into the base portion so that a first portion of the fabric member is encapsulated into the base and a second portion of the fabric member extends from the base to form an external annular fabric band therewith.
